(19) United States
(12) Patent Application Publication
(10) Pub. No.: US 2011/0224488 A1
(43) Pub. Date: Sep. 15, 2011

(54) ENDOSCOPE APPARATUS

(75) Inventor: Jin OHARA, Tokyo (JP)

(73) Assignee: OLYMPUS CORPORATION, Tokyo (JP)

(21) Appl. No.: 13/043,957

(22) Filed: Mar. 9, 2011

(30) Foreign Application Priority Data
Mar. 10, 2010 (JP) JP 2010-052837

(51) Int. Cl. A61B 1/04

(52) U.S. Cl. 600/109

(57) ABSTRACT

An endoscope apparatus includes an endoscope that includes an endoscope tip member at a rigid unit of a tip of the endoscope, and a cooling mechanism for cooling the endoscope tip member. The endoscope tip member includes at least an imaging element unit, an illuminating unit, and a cooling mechanism fitting unit. The cooling mechanism is fit into the cooling mechanism fitting unit.

### CROSS-REFERENCE TO RELATED APPLICATION

The present application is based upon and claims the benefit of priority from the prior Japanese Patent Application No. 2010-52837 filed on Mar. 10, 2010; the entire contents of which are incorporated herein by reference.

### BACKGROUND OF THE INVENTION

1. Field of the Invention

The present invention relates to an endoscope apparatus.

2. Description of the Related Art

An endoscope apparatus is a device that includes an endoscope. The endoscope is inserted inside a test body to, for example, capture images of the interior of the body, acquire live cells from the body, and perform medical treatment on the body. An imaging element and an illuminating unit are provided at a tip of the endoscope. Heat is generated due to a loss arising from conversion between electrical energy and optical energy when the imaging element and the illuminating unit are driven. Generally, the imaging element and the illuminating unit are adversely affected by a temperature rise. For example, a thermal noise is generated in the imaging element and a luminance efficiency of the illuminating unit is reduced with the temperature rise. Therefore, suppressing the temperature rise due to the heat is desirable.

For example, Japanese Patent Application Publication No. 2008-43440 discloses to provide a cooling channel inside an endoscope tip member to suppress the temperature rise. A cooling mechanism for each member such as the imaging element and the illuminating unit is arranged within the endoscope tip member to suppress the temperature rise.

However, when arranging the cooling mechanism that acts as a channel in the endoscope tip member, processing needs to be performed while evading other members provided within the endoscope tip member. Similarly, when arranging the other members by processing the channel first, processing needs to be performed such that these members do not interfere with each other. To make the endoscope tip as small as possible in a radial direction, consideration must be given to a pitch processing tolerance between the cooling mechanism and the other members as there is only a small processing margin. Furthermore, to obtain a safe design value for processing, a larger channel needs to be designed. A larger channel is not suitable for an endoscope having a small diameter. Thus, an adaptability of the conventional technique is low.

In this manner, when arranging the cooling channel inside the endoscope tip member, the cooling channel having a common design (specifications) for all the endoscopes does not work. Because the shape of the cooling channel depends on the shape of the endoscope, a problem of low adaptability arises.

### SUMMARY OF THE INVENTION

The present invention has been made in view of the above discussion. It is an object of the present invention to provide an endoscope apparatus that includes a highly adaptable endoscope cooling mechanism having a shape that is adaptable to endoscopes having different shapes.

To solve the above problems and to achieve the above object, according to an aspect of the present invention there is provided an endoscope apparatus including an endoscope that includes an endoscope tip member in a rigid unit at a tip of the endoscope. The endoscope includes a cooling mechanism for cooling the endoscope tip member. The endoscope tip member includes at least an imaging element unit, an illuminating unit, and a cooling mechanism fitting unit. The cooling mechanism fitting unit is fit into the cooling mechanism.

As another preferred embodiment of the present invention, the cooling mechanism and the cooling mechanism fitting unit are arranged near an outer periphery in a radial direction of the endoscope.

As another preferred embodiment of the present invention, the cooling mechanism fitting unit is arranged more on an inner side in the radial direction than the outer periphery of the endoscope tip member in the radial direction of the endoscope and has at least one or more faces.

As another preferred embodiment of the present invention, the cooling mechanism includes a water cooling unit and a tube.

As another preferred embodiment of the present invention, the water cooling unit is longer in a longitudinal direction of the endoscope than in the radial direction of the endoscope.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWING

FIG. 1 is a drawing that depicts a structure of an endoscope apparatus according to an embodiment of the present invention;

FIG. 2 is a cross-sectional view along a radial axial direction of the endoscope;

FIG. 3 is a drawing explaining a structure of a cooling mechanism;

FIGS. 4A and 4B are cross-sectional views of a water cooling unit; and

FIG. 5 is a cross-sectional view of the endoscope apparatus along a longitudinal direction.

### DETAILED DESCRIPTION OF THE PREFERRED EMBODIMENTS

Exemplary embodiments of an endoscope apparatus according to the present invention are explained in detail below with reference to the accompanying drawings. The present invention is not limited to the following embodiments.

### Embodiment

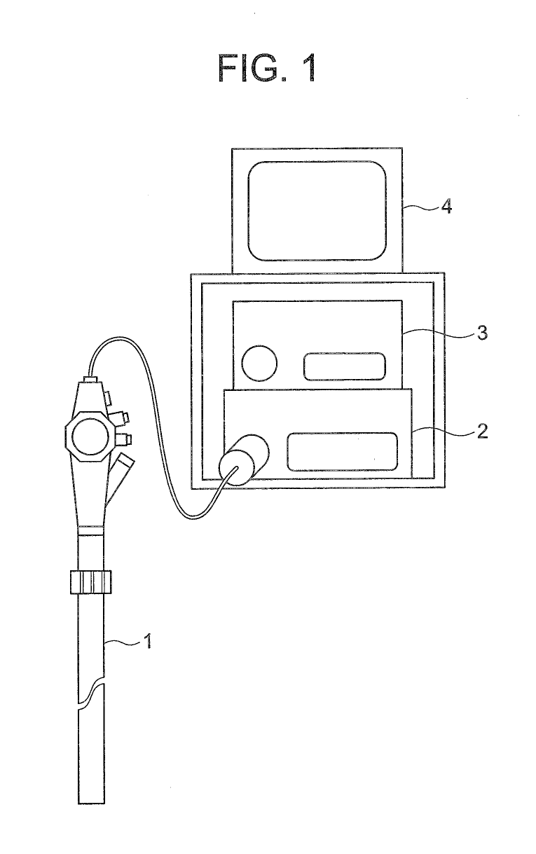

FIG. 1 is a drawing that depicts a structure of an endoscope apparatus according to an embodiment of the present invention. As shown in FIG. 1, the endoscope apparatus includes an endoscope 1, a light source device 2, a video processor 3, and a monitor 4.

The endoscope apparatus is a checking device used to check an internal structure of a test body. The endoscope 1 is inserted inside the test body to capture images of the interior of the body, acquire live cells from the body, and perform medical treatment on the body. The light source device 2, the video processor 3, and the monitor 4 are electrically and mechanically connected to the endoscope 1, and play various roles. The light source device 2 is a device that activates a light source provided in the endoscope 1. The video processor 3 is a device that performs processing of the images received from the endoscope 1, and synchronization and processing of various circuits. The monitor 4 displays the images captured by the endoscope 1.

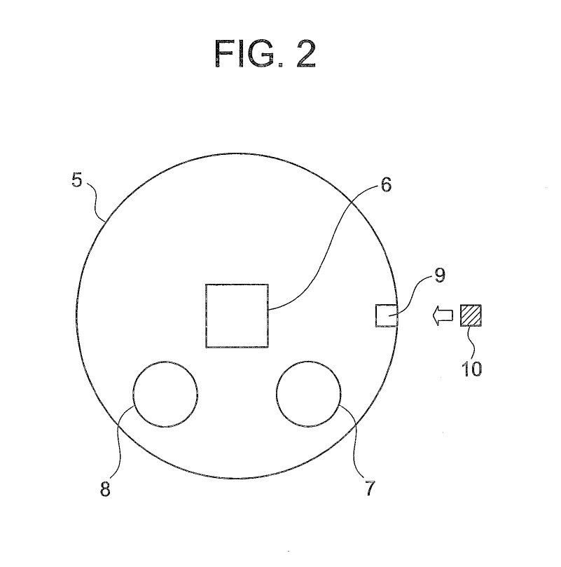

FIG. 2 is a cross-sectional view, from a front side, along a radial direction of the endoscope 1. An endoscope tip member 5 is explained with reference to FIG. 2.

The endoscope tip member 5 of the present embodiment includes the endoscope tip member 5 and a cooling mechanism 10. The endoscope tip member 5 is situated at a tip of the endoscope 1 in a longitudinal direction. The endoscope tip member 5 internally includes an imaging element unit 6, an illuminating unit 7, a forceps unit 8, and a cooling mechanism fitting unit 9.

A shape of the endoscope tip member 5 differs from one endoscope to another endoscope. The endoscope tip member 5 of some endoscopes may not include the forceps unit 8 or it may include components, for example, a built-in water pipe, or functions different from those described above. A member that internally includes at least the imaging element unit 6, the illuminating unit 7, and the cooling mechanism fitting unit 9 is called the endoscope tip member 5. In the embodiment of the present invention, the endoscope tip member 5 that includes the forceps unit 8 in addition to the imaging element unit 6, the illuminating unit 7, and the cooling mechanism fitting unit 9 is explained.

The imaging element unit 6 includes an imaging element, electrical connections or wirings that activate the imaging element, and an optical system such as one or more lenses. The illuminating unit 7 includes a light source that produces a light and a light transmitting member such as an optic fiber that transmits the light up to the tip of the endoscope 1. The light source is, for example, a xenon lamp or a light emitting diode (LED).

Heat is generated due to a loss arising from conversion between electrical energy and optical energy when the imaging element unit 6 and the illuminating unit 7 are driven. The generated heat is transferred to the endoscope tip member 5 and other members. Generally, the imaging element and the light source are adversely affected due to a temperature rise. For example, a thermal noise is generated in the imaging element and a luminance efficiency of the light source is reduced due to the temperature rise. Therefore, suppressing the temperature rise due to the heat is desirable.

The forceps unit 8 is a hole used when introducing a forceps inside the body through the endoscope 1. Because, the forceps moves in and out, the forceps unit 8 is a hollow cavity. The cooling mechanism fitting unit 9 is arranged near an outer periphery of the endoscope tip member 5 in the radial direction of the endoscope 1 to fit the cooling mechanism 10, and it is made by drilling, etc.

The cooling mechanism 10 is fit inside the cooling mechanism fitting unit 9 with an adhesive agent, grease, etc.

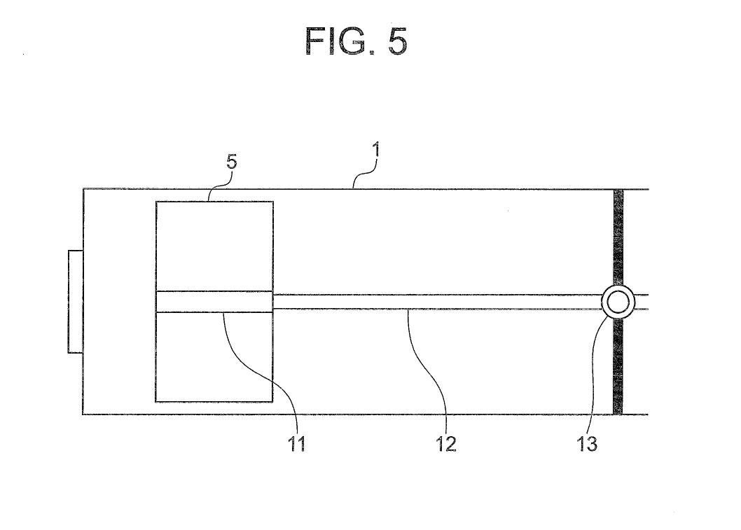

FIG. 5 is a cross-sectional view of the endoscope apparatus in a longitudinal direction. As shown in FIG. 5, the endoscope tip member 5 is arranged within a tip section of the endoscope 1.

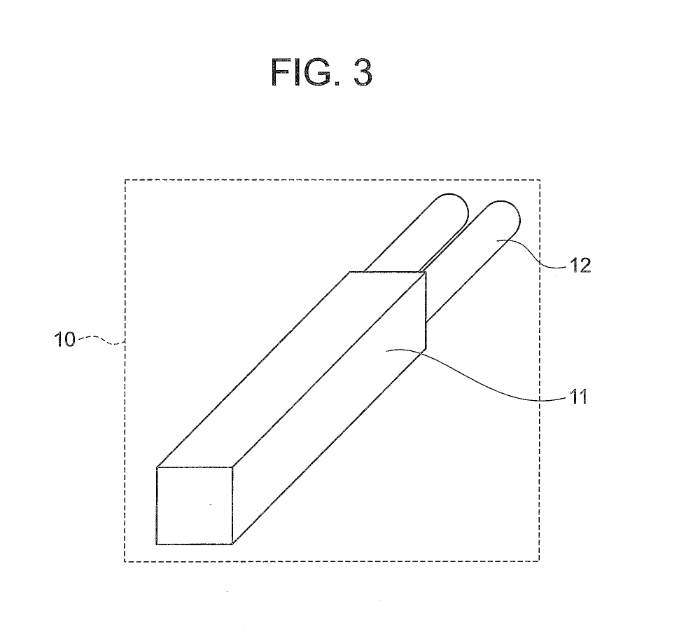

Shapes of the cooling mechanism 10 and the cooling mechanism fitting unit 9 and advantages of the embodiment are explained with reference to FIGS. 3 to 5.

As described above, the cooling mechanism fitting unit 9 is provided near the outer periphery of the endoscope tip member 5 in the radial direction of the endoscope 1.

For comparison purpose, a case is explained in which the cooling mechanism fitting unit 9 is arranged in a portion that is not near the outer periphery of the endoscope tip member 5. For example, when providing the cooling mechanism 10 that acts as a channel in the endoscope tip member 5, the processing needs to be performed by evading the other members arranged within the endoscope tip member 5. Similarly, when providing the other members by processing the channel first, the processing needs to be performed such that these members do not interfere with each other.

For making the endoscope tip member 5 as small as possible in a radial direction, consideration must be given to a pitch processing tolerance between various members as there is only a small processing margin. Furthermore, to obtain a safe design value for processing, a larger channel needs to be designed. A larger channel is not suitable for an endoscope having a small diameter. Thus, an adaptability of the conventional technique is low.

Even if a channel of a smaller size is made, processing with a high aspect ratio becomes extremely difficult, thus increasing a processing cost. This can be considered as an additional disadvantage.

On the other hand, no member can be arranged near the outer periphery of the endoscope tip member 5 in the radial direction of the endoscope 1. This is because, for example, when members for water conveyance, a light source, etc. are provided near the outer periphery, members that seal water and light are required in addition to the endoscope tip member 5 and the diameter of the endoscope tip member 5 needs to be made larger to accommodate these members. Furthermore, it is also desirable to assemble the members as far as possible near the center of the radial direction of the endoscope 1.

In other words, the outer periphery of the endoscope tip member 5 in the radial direction of the endoscope 1 is a dead space for introducing a member. Thus, there is a room for improvement.

In the present embodiment, the cooling mechanism fitting unit 9 is provided near the outer periphery of the endoscope tip member 5 in the radial direction of the endoscope 1 and cooling is performed by using the cooling mechanism 10. The heat generated by various members within the endoscope tip member 5 is not cooled separately for each member; however, the endoscope tip member 5 to which the heat generated by various members is transferred is cooled, thus indirectly cooling the heat generated by the various members.

The cooling mechanism 10 is a separate body than the endoscope tip member 5. Thus, the cooling can be easily performed by arranging the cooling mechanism fitting unit 9 for various endoscopes having the endoscope tip member 5 of different shapes. Furthermore, as described above, fewer members are arranged near the outer periphery of the endoscope tip member 5 in the radial direction. As a result, there are multiple choices for the location of the cooling mechanism fitting unit 9, thus increasing the adaptability.

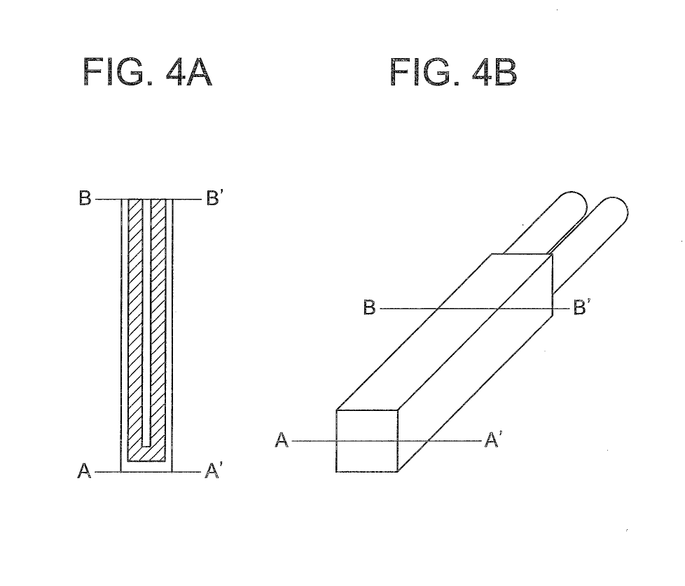

FIG. 3 is a drawing that depicts the cooling mechanism 10. The cooling mechanism 10 includes a water cooling unit 11 and a tube 12. The tube 12 and the water cooling unit 11 are joined with an adhesive agent, etc. FIGS. 4A and 4B are cross-sectional views of the water cooling unit 11. A flow channel is formed near an inner surface of the water cooling unit 11. The endoscope tip member 5 is cooled by passing water as a cooling medium through the flow channel. A fluid can be used as the cooling medium, i.e., gas can also be used.

A size of the cooling mechanism 10 is much smaller than that of the endoscope tip member 5. Therefore, to provide a maximum possible cooling effect, water cooling that has a higher heat transport amount per unit volume is preferable over air cooling.

The tube 12 is coupled to the water cooling unit 11 and it extends to the rear in the longitudinal direction of the endoscope 1. The water used for cooling is circulated through the tube 12; from the rear of the endoscope 1 to the tip of the endoscope 1 and again back to the rear of the endoscope 1. The water is passed through the tube 12 using a pump, etc. The water cooling unit 11 is longer in the longitudinal direction of the endoscope 1 and smaller in the radial direction of the endoscope 1. The water cooling unit 11 is formed in this size for two reasons; first, to avoid interference with the other members inside the endoscope tip member 5 in terms of the space; and second, to obtain as large a flow channel area as possible for water cooling to increase the cooling effect with the cooling mechanism 10 that requires a small space.

Formation of the cooling mechanism 10 includes processes such as plating, cutting, and joining. The cooling mechanism 10 is formed by a processing that is of the order of several 100 micrometers (w). Due to this, forming the cooling mechanism 10 using a top-down method, in which a process of cutting as the target processing is performed, is difficult because this method has limitations in processing accuracy, processing dimensions, etc. On the other hand, if the cooling mechanism 10 is formed using a bottom-up method in which plating is used for creating the entire structure, from the beginning, there is an advantage that the structure can be made more compact as compared to the top-down processing method.

FIG. 5 is the cross-sectional view along the longitudinal direction of the endoscope 1. The water cooling unit 11 is fit into the endoscope tip member 5 and the tube 12 extends from the water cooling unit 11 to the rear in the longitudinal direction of the endoscope 1. The tube 12 passes through side faces of a bend unit 13 and extends to the rear of the endoscope 1. The bend unit 13 is provided to enable bending of the endoscope 1. A tip side of the endoscope 1 located further from the unit 13 is called a rigid unit 14 and a rear side of the endoscope 1 is called a flexible unit 15. Furthermore, the endoscope 1 may not have the bend unit 13. In such an endoscope 1, the entire structure is formed as the rigid unit 14 and the endoscope tip member 5 and the water cooling unit 11 are arranged inside the rigid unit 14.

With the structure described above, highly adaptable cooling can be easily performed by assembling the cooling mechanism 10 and the cooling mechanism fitting unit 9.

Furthermore, because the cooling mechanism 10 is arranged near the outer periphery in the radial direction of the endoscope 1, i.e., in the dead space of the endoscope tip member 5, this structure can be adapted even in a small-diameter endoscope. Furthermore, the processing of the cooling mechanism fitting unit 9 can be easily performed with a drill, etc., thus enhancing the adaptability.

Because the cooling mechanism 10 is a separate body, it can be made compact with a lower processing cost as compared to a case of creating the channel.

As described above, the endoscope apparatus according to the present invention is useful for various endoscope apparatus, and is particularly adapted for the endoscope apparatus that includes the cooling mechanism at the tip of the endoscope.

As an endoscope apparatus according to the present invention, a compact endoscope apparatus is proposed that includes a highly adaptable endoscope cooling mechanism having a shape that is adaptable to endoscopes having different shapes.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. An endoscope apparatus comprising:
an endoscope that includes an endoscope tip member in a rigid unit at a tip of the endoscope, wherein
the endoscope includes a cooling mechanism for cooling the endoscope tip member,
the endoscope tip member includes at least an imaging element unit, an illuminating unit, and a cooling mechanism fitting unit, and
the cooling mechanism fitting unit is fit into the cooling mechanism.

2. The endoscope apparatus according to claim 1, wherein the cooling mechanism and the cooling mechanism fitting unit are arranged near an outer periphery in a radial direction of the endoscope.

3. The endoscope apparatus according to claim 2, wherein the cooling mechanism fitting unit is arranged more on an inner side in the radial direction than the outer periphery of the endoscope tip member in the radial direction of the endoscope and has at least one or more faces.

4. The endoscope apparatus according to claim 3, wherein the cooling mechanism includes a water cooling unit and a tube.

5. The endoscope apparatus according to claim 4, wherein the water cooling unit is longer in a longitudinal direction of the endoscope than in the radial direction of the endoscope.

6. The endoscope apparatus according to claim 2, wherein the cooling mechanism includes a water cooling unit and a tube.

7. The endoscope apparatus according to claim 6, wherein the water cooling unit is longer in a longitudinal direction of the endoscope than in the radial direction of the endoscope.

8. The endoscope apparatus according to claim 1, wherein the cooling mechanism includes a water cooling unit and a tube.

9. The endoscope apparatus according to claim 8, wherein the water cooling unit is longer in a longitudinal direction of the endoscope than in a radial direction of the endoscope.
